(19) United States
(12) Patent Application Publication
(10) Pub. No.: US 2025/0368827 A1
(43) Pub. Date: Dec. 4, 2025

(54) NOVEL PIGMENT DISPERSIONS

(71) Applicant: AMERICAN COLORS, INC., Sandusky, OH (US)

(72) Inventor: Kayla EYER, Sandusky, OH (US)

(21) Appl. No.: 19/224,617

(22) Filed: May 30, 2025

Related U.S. Application Data
(60) Provisional application No. 63/655,339, filed on Jun. 3, 2024.

(51) Int. Cl. C09B 67/20; A61K 8/29; A61K 8/37

(52) U.S. Cl. CPC C09B 67/006; A61K 8/29; A61K 8/375; A61K 2800/30; A61K 2800/43

(57) ABSTRACT

The invention relates to novel pigment dispersions, such as for finished cosmetic and personal care compositions, the pigment dispersions comprising fatty acid esters, such as polyglyceryl fatty acid esters, and methods of making and using the same. In some embodiments, the dispersions are free of alcohols, silicone derivatives, and/or natural oils. Preferably, the pigment dispersion provides a color concentrate having an NOI (natural origin index) value of close to 1 (e.g., according to ISO 16128).

### CROSS-REFERENCE TO RELATED APPLICATIONS

This application is a U.S. non-provisional application, which claims priority to, and the benefit of, U.S. provisional application Ser. No. 63/655,339, filed on Jun. 3, 2024, the contents of which are hereby incorporated by reference in its entirety.

### FIELD

The invention relates to novel pigment dispersions, such as for finished cosmetic and personal care compositions, the pigment dispersions comprising fatty acid esters, such as polyglyceryl fatty acid esters, and methods of making and using the same. In some embodiments, the dispersions are free of alcohols, silicone derivatives, and/or natural oils. Preferably, the pigment dispersion provides a color concentrate having an NOI (natural origin index) value of close to 1 (e.g., according to ISO 16128).

### BACKGROUND

Personal care compositions are compositions suitable for topical application to the human body, such as the skin and hair, for improving appearance and/or cleanliness. Examples of personal care compositions include skin care products (e.g., facial creams, moisturizers, face and body lotions, sunscreens, foundation, mascara, eye-liner, lipsticks, liquid soaps, solid soaps, body washes, cleansers, and the like) and hair care products (e.g., shampoos, conditioners, styling gels and hairsprays). These compositions are often intended to clean and/or to moisturize the skin and hair, and keep them in a smooth condition.

Personal care compositions must be carefully formulated to provide maximum wear and effect, and to avoid incompatibilities between ingredients which can affect stability, storability, and appearance.

Personal care compositions, such as cosmetics and other similar products, are commonly strongly colored. Color may generally be added to otherwise colorless products or composition using either dyes or pigments. Dyes are agents which impart color and which are soluble in the carrier or vehicle used for the composition or product. Because of their solubility, dyes will spontaneously evenly distribute themselves within a liquid carrier, generally with little or no risk of precipitation or heterogeneity. In contrast to dyes, pigments are agents which impart color but are insoluble in the carrier or vehicle used for the composition or product. Because of this insolubility, pigments are not in a dissolved state, but rather, are in a dispersed state, within the liquid carrier or vehicle. Thus, pigment must be dispersed in, and stabilized in, a vehicle, in order to achieve the characteristics that are required for a pigment's intended use, such as color development, opacity, and particle size.

Dispersions often are not inherently stable: the dispersed pigment may agglomerate, precipitate, stratify, or otherwise become heterogeneous, resulting in unacceptable appearance and manufacturing process difficulties (such as clogging of pipes). Significant effort is often necessary to provide a pigment dispersion which is homogenous and physically stable, such as, by adding wetting aids, dispersing aids, and/or anti-settling aids. In addition, from a product quality control perspective, it is necessary, not only that the pigment dispersion be stable, but also that it is reproducible, so that there is consistency in color between batches of product, preferably also with a minimum amount of manufacturing time and labor required.

Essentially, a dispersion wets out a pigment, stabilizing it in a liquid carrier, or vehicle. This can sometimes be achieved by simply mixing the pigment into the carrier, where the carrier has enough dispersive properties to stabilize the solid pigment particles itself. Otherwise, dispersing aids (dispersants, also called surfactants), are necessary to achieve stabilization.

Forming stable pigments dispersions with the desired end-user properties can be difficult. Some pigments may require more or less shear force, wetting agent, or dispersing agent, or combinations thereof, to achieve the desired properties for the application. In some cases, pigments are mixed or dispersed directly into an end application or a portion of the end application formula, which may not fully develop the color, achieve the desired particle (agglomerate) size reduction, or maintain the desired properties over time. In some cases, pigments may be surface treated with additives to aid in the mixing or dispersing directly into an end application, which may add undesired ingredients to the formula. These surface treated pigments may still not fully develop the color, achieve the desired particle (agglomerate) size reduction, or maintain the desired properties over time

Another benefit of using pigment dispersion relates to handling. Pigments are generally solids, often dry, powdery or fine solids, and in bulk, pigments can present handling difficulties and hazards, such as inhalation hazards and flammability hazards, and difficulty measuring quantities and cleaning up residues. From a manufacturing standpoint, it is much safer, and more efficient, to work with a liquid dispersion of pigment, which can easily be incorporated into personal care compositions and other products.

Pigment dispersions solve these problems by pre-dispersing pigments in an environment that is safe and adequate for handling powders into liquid mediums that are both appropriate for the end application and support the particle (agglomerate) size reduction, color development, and stability of the colorant, making them ready to use.

The selection of liquid medium to process the pigments or other powders and deliver the desired properties is critical.

Pigment dispersions are used in many applications, including coatings, plastics, films, composites, adhesives, caulks, cosmetics, and pharmaceuticals. For industrial products, such as coatings, the liquid medium of a dispersion may consist of solvent (e.g., organic solvent), oils, polymers, or combinations thereof, combined with additives such as wetting aids, dispersing aids, surfactants, and suspension aid, as well as pH buffers or modifiers, preservatives, and defoaming/anti-foaming aids if water-containing.

Unlike industrial uses of pigments and pigment dispersions, pigments and pigment dispersions for cosmetic and personal care applications face additional hurdles, including consumer acceptance, health and safety, and regulatory limitations.

Regional governmental and nongovernmental regulatory organizations, as well as consumer advocacy groups, regulate and influence what is used in these products, as well as how they must be prepared. For this reason, the common organic solvents and polymers/binders used in dispersions for industrial applications are mostly excluded from use in cosmetic and pharmaceutical applications. Instead, these applications rely on a growing body of research on materials that are safe for use in that they do not contain chemicals that may be carcinogens, mutagens, reprotoxins, irritants, or cause other long-term or short-term health consequences when used as intended. In recent years, consumer advocacy groups have also pushed cosmetic and pharmaceutical markets toward materials with sustainability claims such as natural, naturally-derived, biodegradable, organic, and sustainably sourced. A similar push has also occurred in the coatings and plastics markets to a lesser extent.

These limitations on the ingredients which are acceptable for use in pigment dispersions for cosmetic, pharmaceutical, and other non-industrial or consumer applications, further increases the difficulty of formulating pigment dispersions for these uses.

Castor oil is a common carrier or vehicle used for cosmetic and personal care pigment dispersions. Castor oil is a natural oil, derived from castor beans, and it consists primarily of triglycerides, mostly ricinoleates. While castor oil is a very good, naturally-derived, dispersion vehicle that is prevalent and inexpensive, it can create dispersions with higher viscosities than desired, and it may cause heavier skin feel and reduced spreadability, and may not be suitable for all emulsion applications. In addition, there is presently a strong consumer interest in “oil-free” compositions, which means compositions free of naturally-derived oils, such as castor oil, castor seed oil, palm oil, jojoba seed oil, sunflower seed oil, coconut oil, palm kernel oil, and the like. At the same time, there is also high consumer interest in naturally-derived ingredients, such as ingredients derived from natural sources and semi-synthetic ingredients, which are chemically modified from the form found in nature.

Existing oil-free pigments dispersions have been made using light-to-medium esters, such as isononyl isononanoate, isopropyl myristate, C12-15 alkyl benzoate, polyglyceryl-6 polyricinoleate, octyldodecyl oleate, other triglycerides such as caprylic/capric triglyceride, or various hydrocarbons. While these carrier vehicles may achieve lower viscosity, lighter skin feel, and may be more suitable for emulsion formulations or “oil-free” formulations, they do not achieve suitable dispersion, color development, and/or dispersion stability characteristics, and some are not naturally-derived. The problem with existing dispersions, particularly pigment dispersions for use in color cosmetics applications, is that no one solution offers all of these benefits: naturally-derived, light skin feel, exceptional dispersion characteristics and color development, low viscosity, shelf stability, “oil-free”, good spreadability, and suitability for emulsion formulations.

Traditional liquid polymers, such as polyethylene glycols, mixed glycol polymers, poloxamers, and silicone polymers (silicones), have had important utility in cosmetic and personal care applications, but for the reasons set forth above, there has been a consumer and market desire to move away from these traditional ingredients.

Therefore, there remains a need for new, stable, pigment dispersion formulations, especially for use in cosmetic and personal are compositions, which are preferably naturally-derived, have light skin feel, exceptional dispersion characteristics and color development, low viscosity, shelf stability, are oil-free, have good spreadability, and are suitable for emulsion formulations.

The present disclosure provides new pigment dispersion formulations, such as for cosmetic and personal care compositions, which comprise fatty acid esters, such as polyglyceryl fatty acid esters, and methods of making and using the same. In some embodiments, the dispersions are free of alcohols, silicone derivatives, and/or natural oils. Preferably, the pigment dispersions provide a color concentrate having an NOI (natural origin index) value of close to 1 (e.g., according to ISO 16128).

### BRIEF SUMMARY

In a first aspect, the present disclosure provides a stable pigment dispersion comprising one or more fatty acid esters, wherein the composition comprises at least one polyglyceryl fatty acid ester. Preferably, the composition is alcohol-free, silicone-free, and/or free of natural oils (e.g., castor oil). Optionally, the compositions may comprise other fatty acid esters, such as fatty alcohol fatty acid esters or mono-, di-, or tri-glycerides.

The inventors have found that compositions according to the present disclosure effectively balances the needs for particle dispersion and color development with the benefits of low viscosity and light skin feel, resulting in a total dispersion formulation that is shelf stable, “oil-free,” and suitable for emulsions, with low tack. Further, the disclosure provides volatile-free dispersions with high compatibility for mixing into oil-free, silicone-free, and alcohol-free mediums, with the potential for cosmetic and personal care compositions with naturally-derived content up to 100%.

In further embodiments, the present disclosure provides methods of making and/or using the pigment dispersions, such as, to make cosmetic or personal care compositions.

Additional features and advantages of the dispersions, composition, and methods disclosed herein will be apparent from the following detailed description.

### DETAILED DESCRIPTION

Although specific embodiments of the present disclosure will now be described with reference to the examples provided herein, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure, and as further defined in the appended claims.

Unless defined otherwise, all technical and scientific terms used herein have the same meanings as commonly understood by one of ordinary skill in the art to which this disclosure belongs.

The compositions disclosed herein are preferably oil-free. As used herein, “oil-free” means free of naturally-derived oils, such as castor oil, castor seed oil, jojoba seed oil, sunflower seed oil, coconut oil, palm kernel oil, peanut oil, soybean oil, grape seed oil, rice bran oil, linseed oil, Brazil nut oil, Buriti oil, Pracaxi oil, and the like. In this context, “oil” refers to natural oils, but not synthetically modified natural oils or compounds or fractions isolated from a natural oil. Synthetic modifications of a natural oil, such as by esterification, render the resultant material not a product-of-nature, and thus, not a natural oil. Similarly, extraction of particular components or mixture of components from a natural oil result in a material that is not itself a natural oil (although it is still naturally-derived, which may be favorable in some respects).

In a first aspect, the present disclosure provides a pigment dispersion (Dispersion 1) comprising at least one polyglyceryl fatty acid ester. Preferably, the dispersion is alcohol-free, silicone-free, and/or free of natural oils (e.g., castor oil). Optionally, the dispersions may comprise other fatty acid esters, such as fatty alcohol fatty acid esters or mono-, di-, or tri-glycerides. In further embodiments of the first aspect, the present disclosure provides:
1.1 Dispersion 1, wherein the polyglyceryl fatty acid ester is a monoester, diester, triester, tetraester, or pentaester of a polyglycerol;
1.2 Dispersion 1, or 1.1, wherein the polyglyceryl fatty acid is a discrete monoester (e.g., of a single fatty acid);
1.3 Dispersion 1, or 1.1, wherein the polyglyceryl fatty acid is a discrete diester (e.g., of a single fatty acid);
1.4 Dispersion 1, or 1.1, wherein the polyglyceryl fatty acid is a discrete triester (e.g., of a single fatty acid);
1.5 Dispersion 1, or 1.1, wherein the polyglyceryl fatty acid is a discrete tetraester (e.g., of a single fatty acid);
1.6 Dispersion 1, or 1.1, wherein the polyglyceryl fatty acid is a discrete pentaester (e.g., of a single fatty acid);
1.7 Dispersion 1, or 1.1, wherein the polyglyceryl fatty acid is a mixed diester (e.g., of two or more fatty acids);
1.8 Dispersion 1, or 1.1, wherein the polyglyceryl fatty acid is a mixed triester (e.g., of two or more fatty acids);
1.9 Dispersion 1, or 1.1, wherein the polyglyceryl fatty acid is a mixed tetraester (e.g., of two or more fatty acids);
1.10 Dispersion 1, or 1.1, wherein the polyglyceryl fatty acid is a mixed pentaester (e.g., of two or more fatty acids);
1.11 Dispersion 1, or 1.1, wherein the polyglyceryl fatty acid is a mixed ester of a fatty acid mixture, e.g., a mixed ester of a vegetable oil-derived fatty acid mixture;
1.12 Dispersion 1, or any of 1.1-1.11, wherein the polyglyceryl fatty acid ester is an ester of a polyglycerol of the following formula, wherein n is an integer from 2 to 20;

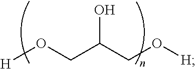

1.13 Dispersion 1.12, wherein n is an integer from 2 to 10;
1.14 Dispersion 1.13, wherein n is an integer selected from 2, 3, 4, 5, 6, 7, 8, 9, and 10;
1.15 Dispersion 1.14, wherein n is an integer selected from 2, 3, 4, 5, and 6;
1.16 Dispersion 1.15, wherein n is an integer selected from 3, 4, 5 and 6;
1.17 Dispersion 1.12, wherein n is 2;
1.18 Dispersion 1.12, wherein n is 3;
1.19 Dispersion 1.12, wherein n is 4;
1.20 Dispersion 1.12, wherein n is 5;
1.21 Dispersion 1.12, wherein n is 6;
1.22 Dispersion 1, or any of 1.1-1.21, wherein the dispersion comprises a mixture of polyglyceryl fatty acid esters having an average n value as set forth in any of 1.12-1.21;
1.23 Dispersion 1 or any of 1.1-1.22, wherein the polyglyceryl fatty acid ester comprises a polyglyceryl-3 fatty acid ester;
1.24 Dispersion 1 or any of 1.1-1.22, wherein the polyglyceryl fatty acid ester comprises a polyglyceryl-4 fatty acid ester;
1.25 Dispersion 1 or any of 1.1-1.22, wherein the polyglyceryl fatty acid ester comprises a polyglyceryl-5 fatty acid ester;
1.26 Dispersion 1 or any of 1.1-1.22, wherein the polyglyceryl fatty acid ester comprises a polyglyceryl-6 fatty acid ester;
1.27 Dispersion 1 or any of 1.1-1.26, wherein the polyglyceryl fatty acid ester is a monoester, diester, triester, tetraester, or pentaester;
1.28 Dispersion 1, or any of 1.1-1.26, wherein the polyglyceryl fatty acid ester is a stearate, isostearate, laurate, oleate, linoleate, linolenate, caprate, caprylate, myristate, ricinoleate, behenate, palmitate, isopalmitate, sebacate, undecylenate, or dilinoleate ester;
1.29 Dispersion 1, or any of 1.1-1.26, wherein the polyglyceryl fatty acid ester is a stearate, isostearate, laurate, oleate, linoleate, linolenate, caprate, caprylate, myristate, ricinoleate, behenate, palmitate, isopalmitate, sebacate, undecylenate, or dilinoleate monoester;
1.30 Dispersion 1, or any of 1.1-1.26, wherein the polyglyceryl fatty acid ester is a diester comprising two or more of stearate, isostearate, laurate, oleate, linoleate, linolenate, caprate, caprylate, myristate, ricinoleate, behenate, palmitate, isopalmitate, sebacate, undecylenate, or dilinoleate ester groups;
1.31 Dispersion 1, or any of 1.1-1.26, wherein the polyglyceryl fatty acid ester is a triester comprising two or more of stearate, isostearate, laurate, oleate, linoleate, linolenate, caprate, caprylate, myristate, ricinoleate, behenate, palmitate, isopalmitate, sebacate, undecylenate, or dilinoleate ester groups;
1.32 Dispersion 1, or any of 1.1-1.26, wherein the polyglyceryl fatty acid ester is a tetraester comprising two or more of stearate, isostearate, laurate, oleate, linoleate, linolenate, caprate, caprylate, myristate, ricinoleate, behenate, palmitate, isopalmitate, sebacate, undecylenate, or dilinoleate ester groups;
1.33 Dispersion 1, or any of 1.1-1.26, wherein the polyglyceryl fatty acid ester is a pentaester comprising two or more of stearate, isostearate, laurate, oleate, linoleate, linolenate, caprate, caprylate, myristate, ricinoleate, behenate, palmitate, isopalmitate, sebacate, undecylenate, or dilinoleate ester groups;
1.34 Dispersion 1, or any of 1.1-1.26, wherein the polyglyceryl fatty acid is an ester derived from the fatty acids of apricot kernel oil, avocado oil, borage seed oil, candelilla oil, jojoba seed oil, rice bran oil, castor oil, linseed oil, macadamia nut oil, olive oil, palm kern oil, palm oil, coconut oil, pumpkin seed oil, safflower seed oil, sesame oil, shea butter, sunflower seed oil, sweet almond oil, watermelon seed oil, or any combination thereof.
1.35 Dispersion 1, or any of 1.1-1.34, wherein the polyglyceryl fatty acid ester is selected from any of the following or a combination thereof: Almond Oil/Polyglyceryl-10 Esters, Apricot Kernel Oil Polyglyceryl-3 Esters, Apricot Kernel Oil Polyglyceryl-4 Esters, Apricot Kernel Oil Polyglyceryl-5 Esters, Apricot Kernel Oil Polyglyceryl-6 Esters, Apricot Kernel Oil Polyglyceryl-10 Esters, Argan Oil Polyglyceryl-6 Esters, Astrocaryum Vulgare Oil Polyglyceryl-6 Esters, Avocado Oil Polyglyceryl-6 Esters, Babassu Oil Polyglyceryl-4 Esters, Babassu Oil Polyglyceryl-6 Esters, Bertholletia Excelsa Seed Oil Polyglyceryl-6 Esters, Borage Seed Oil Polyglyceryl-4 Esters, Borage Seed Oil Polyglyceryl-6 Esters, Candelilla/Jojoba/Rice Bran Polyglyceryl-3 Esters, Caprylic/Capric Glycerides Polyglyceryl-10 Esters, Carapa Guaianensis Oil Polyglyceryl-6 Esters, Castor Oil Polyglyceryl-6 Esters, Cocoa Butter Polyglyceryl-6 Esters, Coconut Oil Polyglyceryl-6 Esters, Coffee Seed Oil Polyglyceryl-6 Esters, Diisostearoyl Polyglyceryl-3 Dimer Dilinoleate, Glyceryl/Polyglyceryl-6 Isostearate/Behenate Esters, Hazelnut Seed Oil Polyglyceryl-6 Esters, Linseed Oil Polyglyceryl-4 Esters, Macadamia Seed Oil Polyglyceryl-6 Esters, Macadamia Seed Oil Polyglyceryl-6 Esters, Behenate Mauritia Flexuosa Seed Oil Polyglyceryl-6 Esters, Olive Oil Polyglyceryl-3 Esters, Olive Oil Polyglyceryl-4 Esters, Olive Oil Polyglyceryl-6 Esters, Palm Kernel Oil Polyglyceryl-4 Esters, Palm Oil Polyglyceryl-3 Esters, Palm Oil Polyglyceryl-4 Esters, Palm Oil Polyglyceryl-5 Esters, Palm Oil Polyglyceryl-6 Esters, Parinari Curatellifolia Oil Polyglyceryl-6 Esters, Pinus Sibirica Seed Oil Polyglyceryl-6 Esters, Polyglyceryl-2 Caprate, Polyglyceryl-2 Caprylate, Polyglyceryl-2 Diisostearate, Polyglyceryl-2 Dioleate, Polyglyceryl-2 Distearate, Polyglyceryl-2 Isopalmitate, Polyglyceryl-2 Isopalmitate/Sebacate, Polyglyceryl-2 Isostearate, Polyglyceryl-2 Laurate, Polyglyceryl-2 Myristate, Polyglyceryl-2 Oleate, Polyglyceryl-2 Palmitate, Polyglyceryl-2 Sesquicaprylate, Polyglyceryl-2 Sesquiisostearate, Polyglyceryl-2 Sesquioleate, Polyglyceryl-2 Sesquistearate, Polyglyceryl-2 Stearate, Polyglyceryl-2 Tetrabehenate/Macadamiate/Sebacate, Polyglyceryl-2 Tetraisostearate, Polyglyceryl-2 Tetraoleate, Polyglyceryl-2 Tetrastearate, Polyglyceryl-2 Triisostearate, Polyglyceryl-3 Beeswax, Polyglyceryl-3 Behenate, Polyglyceryl-3 Caprate, Polyglyceryl-3 Caprylate, Polyglyceryl-3 Cocoate, Polyglyceryl-3 Dicaprate, Polyglyceryl-3 Dicitrate/Stearate, Polyglyceryl-3 Dicocoate, Polyglyceryl-3 Di-Hydroxystearate, Polyglyceryl-3 Diisostearate, Polyglyceryl-3 Dioleate, Polyglyceryl-3 Distearate, Polyglyceryl-3 isostearate, Polyglyceryl-3 Laurate, Polyglyceryl-3 Myristate, Polyglyceryl-3 Oleate, Polyglyceryl-3 Palmitate, Polyglyceryl-3 Pentacaprylate/Caprate, Polyglyceryl-3 Pentaolivate, Polyglyceryl-3 Pentaricinoleate, Polyglyceryl-3 Rice Branate, Polyglyceryl-3 Ricinoleate, Polyglyceryl-3 Soyate/Shea Butterate, Polyglyceryl-3 Stearate, Polyglyceryl-3 Stearate SE, Polyglyceryl-3 Triisostearate, Polyglyceryl-3 Triolivate, Polyglyceryl-4 Almondate/Shea Butterate, Polyglyceryl-4 Caprate, Polyglyceryl-4 Caprylate, Polyglyceryl-4 Caprylate/Caprate, Polyglyceryl-4 Cocoate, Polyglyceryl-4 Dilaurate, Polyglyceryl-4 Distearate, Polyglyceryl-4 Hazelnut sedate, Polyglyceryl-4 Isostearate, Polyglyceryl-4 Isostearate/Laurate, Polyglyceryl-4 Laurate, Polyglyceryl-4 Laurate/Sebacate, Polyglyceryl-4 Laurate/Succinate, Polyglyceryl-4 Oleate, Polyglyceryl-4 Pentaoleate, Polyglyceryl-4 Pentapalmitate/Stearate, Polyglyceryl-4 Pentastearate, Polyglyceryl-4 Punicate, Polyglyceryl-4 Stearate, Polyglyceryl-4 Sweet Almondate, Polyglyceryl-4 Tristearate, Polyglyceryl-5 Caprate, Polyglyceryl-5 Dicaprylate, Polyglyceryl-5 Dilaurate, Polyglyceryl-5 Dioleate, Polyglyceryl-5 Hexastearate, Polyglyceryl-5 Isostearate, Polyglyceryl-5 Laurate, Polyglyceryl-5 Myristate, Polyglyceryl-5 Oleate, Polyglyceryl-5 Pentamyristate, Polyglyceryl-5 Ricinoleate, Polyglyceryl-5 Stearate, Polyglyceryl-5 Tribehenate, Polyglyceryl-5 Triisostearate, Polyglyceryl-5 Trimyristate, Polyglyceryl-5 Trioleate, Polyglyceryl-5 Tristearate, Polyglyceryl-6 Adansonia Digitata Seedate, Polyglyceryl-6 Apricot Kernelate, Polyglyceryl-6 Argan Kernelate, Polyglyceryl-6 Behenate, Polyglyceryl-6 Caprate, Polyglyceryl-6 Caprylate, Polyglyceryl-6 Caprylate/Caprate, Polyglyceryl-6 Citrullus Lanatus Seedate, Polyglyceryl-6 Dicaprate, Polyglyceryl-6 Diisostearate, Polyglyceryl-6 Dioleate, Polyglyceryl-6 Dipalmitate, Polyglyceryl-6 Distearate, Polyglyceryl-6 Heptacaprylate, Polyglyceryl-6 Hexaoleate, Polyglyceryl-6 Hexastearate, Polyglyceryl-6 isostearate, Polyglyceryl-6 Laurate, Polyglyceryl-6 Myristate, Polyglyceryl-6 Octacaprylate, Polyglyceryl-6 Octastearate, Polyglyceryl-6 Oleate, Polyglyceryl-6 Palmitate, Polyglyceryl-6 Palmitate/Succinate, Polyglyceryl-6 Pentacaprylate, Polyglyceryl-6 Pentaoleate, Polyglyceryl-6 Pentaricinoleate, Polyglyceryl-6 Pentastearate, Polyglyceryl-6 Polyricinoleate, Polyglyceryl-6 Ricinoleate, Polyglyceryl-6 Schinziophyton Rautanenii Kemelate, Polyglyceryl-6 Sclerocarya Birrea Seedate, Polyglyceryl-6 Sesquicaprylate, Polyglyceryl-6 Sesquiisostearate, Polyglyceryl-6 Sesquistearate, Polyglyceryl-6 Stearate, Polyglyceryl-6 Tetrabehenate, Polyglyceryl-6 Tetracaprylate, Polyglyceryl-6 Tetraoleate, Polyglyceryl-6 Tricaprylate, Polyglyceryl-6 Trichilia Emetica Seedate, Polyglyceryl-6 Tristearate, Polyglyceryl-6 Undecylenate, Polyglyceryl-6 Ximenia Americana Seedate, Polyglyceryl-8 CI2-20 Acid Ester, Polyglyceryl-8 Decabehenate/Caprate, Polyglyceryl-8 Decaerucate/Decaisostearate/Decaricinoleate, Polyglyceryl-8 Oleate, Polyglyceryl-8 Stearate, Polyglyceryl-10 Apricot Kemelate, Polyglyceryl-10 Behenate/Eicosadioate, Polyglyceryl-10 Caprate, Polyglyceryl-10 Caprylate, Polyglyceryl-10 Caprylate/Caprate, Polyglyceryl-10 Cocoate, Polyglyceryl-10 Decaethylhexanoate, Polyglyceryl-10 Decahydroxystearate, Polyglyceryl-10 Decaisostearate, Polyglyceryl-10 Decalinoleate, Polyglyceryl-10 Decamacadamiate, Polyglyceryl-10 Decaoleate, Polyglyceryl-10 Decastearate, Polyglyceryl-10 Dicocoate, Polyglyceryl-10 Didecanoate, Polyglyceryl-10 Diisostearate, Polyglyceryl-10 Dilaurate, Polyglyceryl-10 Dimyristate, Polyglyceryl-10 Dioleate, Polyglyceryl-10 Dipalmitate, Polyglyceryl-10 Distearate, Polyglyceryl-10 Dodecabehenate, Polyglyceryl-10 Dodecacaprate, Polyglyceryl-10 Dodecacaprylate, Polyglyceryl-10 Dodeca-Caprylate/Caprate, Polyglyceryl-10 Eicosanedioate/Tetradecanedioate, Polyglyceryl-10 Hepta(Behenate/Stearate), Polyglyceryl-10 Heptahydroxystearate, Polyglyceryl-10 Heptaoleate, Polyglyceryl-10 Heptastearate, Polyglyceryl-10 Hexaerucate, Polyglyceryl-10 Hexaisostearate, Polyglyceryl-10 Hexaoleate, Polyglyceryl-10 Hydroxystearate/Stearate/Eieosadioate, Polyglyceryl-10 Isostearate, Polyglyceryl-10 Laurate, Polyglyceryl-10 Linoleate, Polyglyceryl-10 Mono/Dioleate, Polyglyceryl-10 Myristate, Polyglyceryl-10 Nonaerucate, Polyglyceryl-10 Nonaisostearate, Polyglyceryl-10 Oleate, Polyglyceryl-10 Palmate, Polyglyceryl-10 Palmitate, Polyglyceryl-10 Pentacaprylate, Polyglyceryl-10 Pentahydroxystearate, Polyglyceryl-10 Pentaisostearate, Polyglyceryl-10 Pentalaurate, Polyglyceryl-10 Pentalinoleate, Polyglyceryl-10, Pentaoleate, Polyglyceryl-10 Pentaricinoleate, Polyglyceryl-10 Pentastearate, Polyglyceryl-10 Sesquistearate, Polyglyceryl-10 Stearate, Polyglyceryl-10 Tetradecanedioate, Polyglyceryl-10 Tetralaurate, Polyglyceryl-10 Tetraoleate, Polyglyceryl-10 Tricocoate, Polyglyceryl-10 Tridecanoate, Polyglyceryl-10 Trierucate, Polyglyceryl-10 Triisostearate, Polyglyceryl-10 Trilaurate, Polyglyceryl-10 Trioleate, Polyglyceryl-10 Tristearate, Polyglyceryl-10 Undecylenate, Polyglyceryl-15 Diisostearate, Polyglyceryl-20 Docosabehenate/lsostearate, Polyglyceryl-20 Docosabehenate/Laurate, Polyglyceryl-20 Docosabehenate/Oleate, Polyglyceryl-20 Heptacaprylate, Polyglyceryl-20 Heptadecabehenate/Laurate, Polyglyceryl-20 Hexacaprylate, Polyglyceryl-20 Octadecabehenate/Laurate, Polyglyceryl-20 Octaisononanoate, Pumpkin Seed Oil Polyglyceryl-4 Esters, Pumpkin Seed Oil Polyglyceryl-4 Esters Succinate, Rice Bran Oil Polyglyceryl-3 Esters, Rosa Rubiginosa Seed Oil Polyglyceryl-6 Esters, Safflower Seed Oil Polyglyceryl-6 Esters, Schinziophyton Rautanenii Kernel Oil Polyglyceryl-6 Esters, Sclerocarya Birrea Seed Oil Polyglyceryl-6 Esters, Sclerocarya Birrea Seed Oil Polyglyceryl-10 Esters, Sesame Oil Polyglyceryl-6 Esters, Shea Butter Polyglyceryl-3 Esters, Shea Butter Polyglyceryl-4 Esters, Shea Butter Polyglyceryl-6 Esters, Soybean Oil Polyglyceryl-6 Esters, Sunflower Seed Oil Polyglyceryl-3 Esters, Sunflower Seed Oil Polyglyceryl-4 Esters, Sunflower Seed Oil Polyglyceryl-5 Esters, Sunflower Seed Oil Polyglyceryl-6 Esters, Sunflower Seed Oil Polyglyceryl-10 Esters, Sweet Almond Oil Polyglyceryl-4 Esters, Sweet Almond Oil Polyglyceryl-6 Esters, Theobroma Grandiflorum Seed Butter Polyglyceryl-6 Esters, Trichilia Emetica Seed Oil Polyglyceryl-6 Esters, Triisostearoyl Polyglyceryl-3 Dimer Dilinoleate, Watermelon Seed Oil Polyglyceryl-6 Esters, Watermelon Seed Oil Polyglyceryl-10 Esters, and Ximenia Americana Seed Oil Polyglyceryl-6 Esters;
1.36 Dispersion 1, or any of 1.1-1.34, wherein the polyglyceryl fatty acid ester is selected from any of the following or a combination thereof: Diisostearoyl Polyglyceryl-3 Dimer Dilinoleate, Glyceryl/Polyglyceryl-6 Isostearate/Behenate Esters, Polyglyceryl-2 Caprate, Polyglyceryl-2 Caprylate, Polyglyceryl-2 Diisostearate, Polyglyceryl-2 Dioleate, Polyglyceryl-2 Distearate, Polyglyceryl-2 Isopalmitate, Polyglyceryl-2 Isopalmitate/Sebacate, Polyglyceryl-2 Isostearate, Polyglyceryl-2 Laurate, Polyglyceryl-2 Myristate, Polyglyceryl-2 Oleate, Polyglyceryl-2 Palmitate, Polyglyceryl-2 Sesquicaprylate, Polyglyceryl-2 Sesquiisostearate, Polyglyceryl-2 Sesquioleate, Polyglyceryl-2 Sesquistearate, Polyglyceryl-2 Stearate, Polyglyceryl-2 Tetrabehenate/Macadamiate/Sebacate, Polyglyceryl-2 Tetraisostearate, Polyglyceryl-2 Tetraoleate, Polyglyceryl-2 Tetrastearate, Polyglyceryl-2 Triisostearate, Polyglyceryl-3 Behenate, Polyglyceryl-3 Caprate, Polyglyceryl-3 Caprylate, Polyglyceryl-3 Dicaprate, Polyglyceryl-3 Dicitrate/Stearate, Polyglyceryl-3 Di-Hydroxystearate, Polyglyceryl-3 Diisostearate, Polyglyceryl-3 Dioleate, Polyglyceryl-3 Distearate, Polyglyceryl-3 Isostearate, Polyglyceryl-3 Laurate, Polyglyceryl-3 Myristate, Polyglyceryl-3 Oleate, Polyglyceryl-3 Palmitate, Polyglyceryl-3 Pentacaprylate/Caprate, Polyglyceryl-3 Pentaricinoleate, Polyglyceryl-3 Ricinoleate, Polyglyceryl-3 Stearate, Polyglyceryl-3 Stearate SE, Polyglyceryl-3 Triisostearate, Polyglyceryl-4 Caprate, Polyglyceryl-4 Caprylate, Polyglyceryl-4 Caprylate/Caprate, Polyglyceryl-4 Dilaurate, Polyglyceryl-4 Distearate, Polyglyceryl-4 Isostearate, Polyglyceryl-4 Isostearate/Laurate, Polyglyceryl-4 Laurate, Polyglyceryl-4 Laurate/Sebacate, Polyglyceryl-4 Laurate/Succinate, Polyglyceryl-4 Oleate, Polyglyceryl-4 Pentaoleate, Polyglyceryl-4 Pentapalmitate/Stearate, Polyglyceryl-4 Pentastearate, Polyglyceryl-4 Punicate, Polyglyceryl-4 Stearate, Polyglyceryl-4 Tristearate, Polyglyceryl-5 Caprate, Polyglyceryl-5 Dicaprylate, Polyglyceryl-5 Dilaurate, Polyglyceryl-5 Dioleate, Polyglyceryl-5 Hexastearate, Polyglyceryl-5 Isostearate, Polyglyceryl-5 Laurate, Polyglyceryl-5 Myristate, Polyglyceryl-5 Oleate, Polyglyceryl-5 Pentamyristate, Polyglyceryl-5 Ricinoleate, Polyglyceryl-5 Stearate, Polyglyceryl-5 Tribehenate, Polyglyceryl-5 Triisostearate, Polyglyceryl-5 Trimyristate, Polyglyceryl-5 Trioleate, Polyglyceryl-5 Tristearate, Polyglyceryl-6 Behenate, Polyglyceryl-6 Caprate, Polyglyceryl-6 Caprylate, Polyglyceryl-6 Caprylate/Caprate, Polyglyceryl-6 Dicaprate, Polyglyceryl-6 Diisostearate, Polyglyceryl-6 Dioleate, Polyglyceryl-6 Dipalmitate, Polyglyceryl-6 Distearate, Polyglyceryl-6 Heptacaprylate, Polyglyceryl-6 Hexaoleate, Polyglyceryl-6 Hexastearate, Polyglyceryl-6 isostearate, Polyglyceryl-6 Laurate, Polyglyceryl-6 Myristate, Polyglyceryl-6 Octacaprylate, Polyglyceryl-6 Octastearate, Polyglyceryl-6 Oleate, Polyglyceryl-6 Palmitate, Polyglyceryl-6 Palmitate/Succinate, Polyglyceryl-6 Pentacaprylate, Polyglyceryl-6 Pentaoleate, Polyglyceryl-6 Pentaricinoleate, Polyglyceryl-6 Pentastearate, Polyglyceryl-6 Polyricinoleate, Polyglyceryl-6 Ricinoleate, Polyglyceryl-6 Sesquicaprylate, Polyglyceryl-6 Sesquiisostearate, Polyglyceryl-6 Sesquistearate, Polyglyceryl-6 Stearate, Polyglyceryl-6 Tetrabehenate, Polyglyceryl-6 Tetracaprylate, Polyglyceryl-6 Tetraoleate, Polyglyceryl-6 Tricaprylate, Polyglyceryl-6 Tristearate, Polyglyceryl-6 Undecylenate, Polyglyceryl-8 Decabehenate/Caprate, Polyglyceryl-8 Decaerucate/Decaisostearate/Decaricinoleate, Polyglyceryl-8 Oleate, Polyglyceryl-8 Stearate, Polyglyceryl-10 Behenate/Eicosadioate, Polyglyceryl-10 Caprate, Polyglyceryl-10 Caprylate, Polyglyceryl-10 Caprylate/Caprate, Polyglyceryl-10 Decaethylhexanoate, Polyglyceryl-10 Decahydroxystearate, Polyglyceryl-10 Decaisostearate, Polyglyceryl-10 Decalinoleate, Polyglyceryl-10 Decamacadamiate, Polyglyceryl-10 Decaoleate, Polyglyceryl-10 Decastearate, Polyglyceryl-10 Dicocoate, Polyglyceryl-10 Didecanoate, Polyglyceryl-10 Diisostearate, Polyglyceryl-10 Dilaurate, Polyglyceryl-10 Dimyristate, Polyglyceryl-10 Dioleate, Polyglyceryl-10 Dipalmitate, Polyglyceryl-10 Distearate, Polyglyceryl-10 Dodecabehenate, Polyglyceryl-10 Dodecacaprate, Polyglyceryl-10 Dodecacaprylate, Polyglyceryl-10 Dodeca-Caprylate/Caprate, Polyglyceryl-10 Eicosanedioate/Tetradecanedioate, Polyglyceryl-10 Hepta(Behenate/Stearate), Polyglyceryl-10 Heptahydroxystearate, Polyglyceryl-10 Heptaoleate, Polyglyceryl-10 Heptastearate, Polyglyceryl-10 Hexaerucate, Polyglyceryl-10 Hexaisostearate, Polyglyceryl-10 Hexaoleate, Polyglyceryl-10 Hydroxystearate/Stearate/Eieosadioate, Polyglyceryl-10 Isostearate, Polyglyceryl-10 Laurate, Polyglyceryl-10 Linoleate, Polyglyceryl-10 Mono/Dioleate, Polyglyceryl-10 Myristate, Polyglyceryl-10 Nonaerucate, Polyglyceryl-10 Nonaisostearate, Polyglyceryl-10 Oleate, Polyglyceryl-10 Palmate, Polyglyceryl-10 Palmitate, Polyglyceryl-10 Pentacaprylate, Polyglyceryl-10 Pentahydroxystearate, Polyglyceryl-10 Pentaisostearate, Polyglyceryl-10 Pentalaurate, Polyglyceryl-10 Pentalinoleate, Polyglyceryl-10 Pentaoleate, Polyglyceryl-10 Pentaricinoleate, Polyglyceryl-10 Pentastearate, Polyglyceryl-10 Sesquistearate, Polyglyceryl-10 Stearate, Polyglyceryl-10 Tetradecanedioate, Polyglyceryl-10 Tetralaurate, Polyglyceryl-10 Tetraoleate, Polyglyceryl-10 Tridecanoate, Polyglyceryl-10 Trierucate, Polyglyceryl-10 Triisostearate, Polyglyceryl-10 Trilaurate, Polyglyceryl-10 Trioleate, Polyglyceryl-10 Tristearate, Polyglyceryl-10 Undecylenate, Polyglyceryl-15 Diisostearate, Polyglyceryl-20 Docosabehenate/lsostearate, Polyglyceryl-20 Docosabehenate/Laurate, Polyglyceryl-20 Docosabehenate/Oleate, Polyglyceryl-20 Heptacaprylate, Polyglyceryl-20 Heptadecabehenate/Laurate, Polyglyceryl-20 Hexacaprylate, Polyglyceryl-20 Octadecabehenate/Laurate, Polyglyceryl-20 Octaisononanoate, and Triisostearoyl Polyglyceryl-3 Dimer Dilinoleate;
1.37 Dispersion 1, or any of 1.1-1.34, wherein the polyglyceryl fatty acid ester is selected from any of the following or a combination thereof: Diisostearoyl Polyglyceryl-3 Dimer Dilinoleate, Polyglyceryl-2 Caprate, Polyglyceryl-2 Caprylate, Polyglyceryl-2 Diisostearate, Polyglyceryl-2 Dioleate, Polyglyceryl-2 Distearate, Polyglyceryl-2 Isopalmitate, Polyglyceryl-2 Isopalmitate/Sebacate, Polyglyceryl-2 Isostearate, Polyglyceryl-2 Laurate, Polyglyceryl-2 Myristate, Polyglyceryl-2 Oleate, Polyglyceryl-2 Palmitate, Polyglyceryl-2 Sesquicaprylate, Polyglyceryl-2 Sesquiisostearate, Polyglyceryl-2 Sesquioleate, Polyglyceryl-2 Sesquistearate, Polyglyceryl-2 Stearate, Polyglyceryl-2 Tetrabehenate/Macadamiate/Sebacate, Polyglyceryl-2 Tetraisostearate, Polyglyceryl-2 Tetraoleate, Polyglyceryl-2 Tetrastearate, Polyglyceryl-2 Triisostearate, Polyglyceryl-3 Behenate, Polyglyceryl-3 Caprate, Polyglyceryl-3 Caprylate, Polyglyceryl-3 Dicaprate, Polyglyceryl-3 Dicitrate/Stearate, Polyglyceryl-3 Di-Hydroxystearate, Polyglyceryl-3 Diisostearate, Polyglyceryl-3 Dioleate, Polyglyceryl-3 Distearate, Polyglyceryl-3 Isostearate, Polyglyceryl-3 Laurate, Polyglyceryl-3 Myristate, Polyglyceryl-3 Oleate, Polyglyceryl-3 Palmitate, Polyglyceryl-3 Pentacaprylate/Caprate, Polyglyceryl-3 Pentaricinoleate, Polyglyceryl-3 Ricinoleate, Polyglyceryl-3 Stearate, Polyglyceryl-3 Stearate SE, Polyglyceryl-3 Triisostearate, Polyglyceryl-4 Caprate, Polyglyceryl-4 Caprylate, Polyglyceryl-4 Caprylate/Caprate, Polyglyceryl-4 Dilaurate, Polyglyceryl-4 Distearate, Polyglyceryl-4 Isostearate, Polyglyceryl-4 Isostearate/Laurate, Polyglyceryl-4 Laurate, Polyglyceryl-4 Laurate/Sebacate, Polyglyceryl-4 Laurate/Succinate, Polyglyceryl-4 Oleate, Polyglyceryl-4 Pentaoleate, Polyglyceryl-4 Pentapalmitate/Stearate, Polyglyceryl-4 Pentastearate, Polyglyceryl-4 Punicate, Polyglyceryl-4 Stearate, Polyglyceryl-4 Tristearate, Polyglyceryl-5 Caprate, Polyglyceryl-5 Dicaprylate, Polyglyceryl-5 Dilaurate, Polyglyceryl-5 Dioleate, Polyglyceryl-5 Hexastearate, Polyglyceryl-5 Isostearate, Polyglyceryl-5 Laurate, Polyglyceryl-5 Myristate, Polyglyceryl-5 Oleate, Polyglyceryl-5 Pentamyristate, Polyglyceryl-5 Ricinoleate, Polyglyceryl-5 Stearate, Polyglyceryl-5 Tribehenate, Polyglyceryl-5 Triisostearate, Polyglyceryl-5 Trimyristate, Polyglyceryl-5 Trioleate, Polyglyceryl-5 Tristearate, Polyglyceryl-6 Behenate, Polyglyceryl-6 Caprate, Polyglyceryl-6 Caprylate, Polyglyceryl-6 Caprylate/Caprate, Polyglyceryl-6 Dicaprate, Polyglyceryl-6 Diisostearate, Polyglyceryl-6 Dioleate, Polyglyceryl-6 Dipalmitate, Polyglyceryl-6 Distearate, Polyglyceryl-6 Heptacaprylate, Polyglyceryl-6 Hexaoleate, Polyglyceryl-6 Hexastearate, Polyglyceryl-6 isostearate, Polyglyceryl-6 Laurate, Polyglyceryl-6 Myristate, Polyglyceryl-6 Octacaprylate, Polyglyceryl-6 Octastearate, Polyglyceryl-6 Oleate, Polyglyceryl-6 Palmitate, Polyglyceryl-6 Palmitate/Succinate, Polyglyceryl-6 Pentacaprylate, Polyglyceryl-6 Pentaoleate, Polyglyceryl-6 Pentaricinoleate, Polyglyceryl-6 Pentastearate, Polyglyceryl-6 Polyricinoleate, Polyglyceryl-6 Ricinoleate, Polyglyceryl-6 Sesquicaprylate, Polyglyceryl-6 Sesquiisostearate, Polyglyceryl-6 Sesquistearate, Polyglyceryl-6 Stearate, Polyglyceryl-6 Tetrabehenate, Polyglyceryl-6 Tetracaprylate, Polyglyceryl-6 Tetraoleate, Polyglyceryl-6 Tricaprylate, Polyglyceryl-6 Tristearate, Polyglyceryl-6 Undecylenate, and Triisostearoyl Polyglyceryl-3 Dimer Dilinoleate;
1.38 Dispersion 1, or any of 1.1-1.34, wherein the polyglyceryl fatty acid ester is selected from any of the following or a combination thereof: Diisostearoyl Polyglyceryl-3 Dimer Dilinoleate, Polyglyceryl-3 Behenate, Polyglyceryl-3 Caprate, Polyglyceryl-3 Caprylate, Polyglyceryl-3 Dicaprate, Polyglyceryl-3 Dicitrate/Stearate, Polyglyceryl-3 Di-Hydroxystearate, Polyglyceryl-3 Diisostearate, Polyglyceryl-3 Dioleate, Polyglyceryl-3 Distearate, Polyglyceryl-3 Isostearate, Polyglyceryl-3 Laurate, Polyglyceryl-3 Myristate, Polyglyceryl-3 Oleate, Polyglyceryl-3 Palmitate, Polyglyceryl-3 Pentacaprylate/Caprate, Polyglyceryl-3 Pentaricinoleate, Polyglyceryl-3 Ricinoleate, Polyglyceryl-3 Stearate, Polyglyceryl-3 Stearate SE, Polyglyceryl-3 Triisostearate, and Triisostearoyl Polyglyceryl-3 Dimer Dilinoleate;
1.39 Dispersion 1, or any of 1.1-1.34, wherein the polyglyceryl fatty acid ester is selected from Diisostearoyl Polyglyceryl-3 Dimer Dilinoleate, and Triisostearoyl Polyglyceryl-3 Dimer Dilinoleate;
1.40 Dispersion 1, or any of 1.1-1.39, wherein the dispersion further comprises one or more other fatty acid esters;
1.41 Dispersion 1.40, wherein the one or more other fatty acid esters comprise one or more fatty alcohol fatty acid esters or mono-, di-, or tri-glycerides;
1.42 Dispersion 1.41, wherein the one or more other fatty acid esters comprise one or more of isononyl isononanoate, decyl oleate, isostearyl laurate, caprylic/capric triglyceride, diisostearoyl dimer dilinoleate, or a C12-C15 fatty acid benzoate;
1.43 Dispersion 1.41, wherein the one or more other fatty acid esters comprise one or more of isononyl isononanoate, decyl oleate, isostearyl laurate, caprylic/capric triglyceride, and diisostearoyl dimer dilinoleate;
1.44 Dispersion 1.41, wherein the one or more other fatty acid esters comprise one or more of isononyl isononanoate and decyl oleate;
1.45 Dispersion 1.41, wherein the one or more other fatty acid esters comprises decyl oleate;
1.46 Dispersion 1, or any of 1.1-1.45, wherein the dispersion comprises a combination of Diisostearoyl Polyglyceryl-3 Dimer Dilinoleate, Triisostearoyl Polyglyceryl-3 Dimer Dilinoleate, isostearyl isostearate, decyl oleate, Polyglyceryl-3 Laurate, and caprylic/caprate triglyceride, or a combination thereof;
1.47 Dispersion 1.46, wherein the dispersion comprises a combination of Diisostearoyl Polyglyceryl-3 Dimer Dilinoleate and decyl oleate;
1.48 Dispersion 1.46, wherein the dispersion comprises a combination of Triisostearoyl Polyglyceryl-3 Dimer Dilinoleate and decyl oleate;
1.49 Dispersion 1.46, wherein the dispersion comprises a combination of Diisostearoyl Polyglyceryl-3 Dimer Dilinoleate and isostearyl isostearate;
1.50 Dispersion 1.46, wherein the dispersion comprises a combination of Triisostearoyl Polyglyceryl-3 Dimer Dilinoleate and isostearyl isostearate;
1.51 Dispersion 1.46, wherein the dispersion comprises a combination of Polyglyceryl-3 Laurate, and caprylic/caprate triglyceride;
1.52 Dispersion 1, or any of 1.1-1.51, wherein the dispersion further comprises one or more wetting, dispersing, and/or anti-settling aids (e.g., polyhydroxystearic acid, trihydroxystearin, stearoyl glutamic acid, isopropyl titanium triisostearate, stearic acid, dilinoleic acid, sorbitan oleate, ethyl cellulose, and/or disodium stearoyl glutamate);
1.53 Dispersion 1, or any of 1.1-1.51, wherein the dispersion does not comprise one or more wetting, dispersing, and/or anti-settling aids (e.g., wherein the dispersion does not comprise one or more of stearoyl glutamic acid, isopropyl titanium triisostearate, stearic acid, dilinoleic acid, sorbitan oleate, ethyl cellulose, and disodium stearoyl glutamate);
1.54 Dispersion 1, or any of 1.1-1.52, wherein the dispersion further comprises polyhydroxystearic acid, e.g., in an amount of 0.1-10% by weight (e.g., 0.1-8%, or 0.1-6%, or 0.1-4%, or 0.1-3%, or 0.5-10%, or 0.5-8%, or 0.5-6%, or 0.5-4%, or 0.5-3%, or 1-10%, or 1-8%, or 1-6%, or 1-4%, or 1-3%, or 2-10%, or 2-8%, or 2-6%, or 2-4%, or 4-10%, or 4-8%, or 4-6%, or 6-10%, or 6-8%, or 8-10%;
1.55 Dispersion 1, or any of 1.1-1.52, wherein the dispersion further comprises trihydroxystearin, e.g., in an amount of 0.1-5% by weight (e.g., 0.1-4%, or 0.1-3%, or 0.1-2%, or 0.1-1%, or 0.5-5%, or 0.5-4%, or 0.5-3%, or 0.5-2%, or 0.5-1%);
1.56 Dispersion 1, or any of 1.1-1.55, wherein the dispersion is alcohol-free;
1.57 Dispersion 1, or any of 1.1-1.56, wherein the dispersion is silicone-free;
1.58 Dispersion 1, or any of 1.1-1.57, wherein the dispersion is free of natural oils (e.g., castor oil or castor seed oil);
1.59 Dispersion 1, or any of 1.1-1.58, wherein the dispersion comprises from 20-90% by weight of fatty acid esters (e.g., the polyglyceryl fatty acid ester and any other fatty acid esters together), e.g., 20-80%, or 20-70%, or 20-60%, or 20-50%, or 30-80%, or 30-70%, or 30-60%, or 30-50%, or 40-80%, or 40-70%, or 40-60%;
1.60 Dispersion 1, or any of 1.1-1.59, wherein the dispersion comprises from 5-50% by weight of polyglyceryl fatty acid ester, e.g., 5-50%, or 5-40%, or 5-30%, or 5-25%, or 5-20%, or 5-15%, or 10-50%, or 10-40%, or 10-30%, or 10-25%, or 10-20%, or 10-15%, or 15-50%, or 15-40%, or 15-30%, or 15-25%, or 15-20%, or 20-50%, or 20-40%, or 20-30%, or 20-25%;
1.61 Dispersion 1, or any of 1.1-1.59, wherein the dispersion comprises from 10-70% by weight of one or more other fatty acid esters (e.g., decyl oleate, isostearyl isostearate, or triglycerides), e.g., 10-60%, or 10-50%, or 10-40%, or 10-30%, or 20-70%, or 20-60%, or 20-50%, or 20-40%, or 30-70%, or 30-60%, or 30-50%, or 30-40%, or 40-70%, or 40-60%, or 40-50%, or 50-70%, or 50-60%, or 60-70%;
1.62 Dispersion 1, or any of 1.1-1.61, wherein the dispersion is a non-aqueous dispersion;
1.63 Dispersion 1, or any of 1.1-1.62, wherein the pigment is dispersed homogenously in the grinding vehicle and/or carrier;
1.64 Dispersion 1, or any of 1.1-1.63, wherein the pigment is selected from iron oxide (e.g., red iron oxide, yellow iron oxide, black iron oxide or a mixture thereof), titanium dioxide, zinc oxide, bismuth oxychloride, ultramarines (e.g., ultramarine blue, or ultramarine violet), manganese violet, mica, carbon black (e.g., Black 2), lakes (e.g., Red 6 lake, Red 7 lake, Red 21 Lake, Red 27 Lake, Red 28 Lake, Red 30 Lake, Red 33 Lake, Red 34 Lake, Red 36 Lake, Red 40 Lake, Blue 1 Lake, Yellow 5 Lake, Yellow 6 Lake, or Yellow 10 Lake), or any combination thereof;
1.65 Dispersion 1, or any of 1.1-1.64, wherein the pigments are untreated and/or uncoated, and optionally wherein the pigments are cosmetic-grade;
1.66 Dispersion 1, or any of 1.1-1.65, wherein the dispersion comprises 40% or more of pigments, by weight of the dispersion, e.g., 50% or more, or 60% or more, or 70% or more, or 80% or more, or 90% or more, or 95% or more;
1.67 Dispersion 1, or any of 1.1-1.65, wherein the dispersion comprises 40 to 80% of the pigment or pigments, by weight of the dispersion, e.g., 45 to 80%, or 50 to 80%, or 55 to 80%, or 60 to 80%, or 65 to 80%, or 70 to 80%, or 75 to 80%, or 40 to 70%, or 45 to 70%, or 50 to 70%, or 55 to 70%, or 60% to 70%, or 65 to 70%, or 40 to 65%, or 45 to 65%, or 50 to 65%, or 55 to 65%, or 60 to 65%, or 40 to 60%, or 45 to 65%, or 50 to 65%, or 55 to 65%, or 60 to 65%, or 40 to 60%, or 45 to 60%, or 50 to 60%, or 55 to 60%, or 40 to 55%, or 45 to 55%, or 50 to 55%, or 40 to 50%, or 45 to 50%;
1.68 Dispersion 1, or any of 1.1-1.65, wherein the dispersion comprises 5% or more of the pigment or pigments, by weight of the dispersion, e.g., 10% or more, or 20% or more, or 30% or more;
1.69 Dispersion 1, or any of 1.1-1.65, wherein the dispersion comprises 5 to 40% of the pigment or pigments, by weight of the dispersion, e.g., 5 to 35%, or 5 to 30%, or 5 to 25%, or 5 to 20%, or 5 to 15%, or 5 to 10%, or 10 to 40%, or 10 to 35%, or 10 to 30%, or 10 to 25% or 10 to 20%, or 10 to 15%, or 15 to 40%, or 15 to 30%, or 15 to 25%, or 15 to 20%, or 20 to 40%, or 20 to 35%, or 20 to 30%, or 20 to 15%, or 25 to 40%, or 25 to 35%, or 25 to 30%, or 30 to 40%, or 30 to 35%, or 25 to 40%;
1.70 Dispersion 1, or any of 1.1-1.69, wherein the dispersion is free of any one or more of: surfactants, emulsifiers, emollients, humectants, preservatives, stabilizers, rheological additives, antioxidants, thickeners, and hydrocarbons. 1.71 Dispersion 1, or any of 1.1-1.69, wherein the dispersion consists essentially of the pigment, the polyglyceryl fatty acid ester, one or more other fatty acid esters, and optionally polyhydroxystearic acid or trihydroxystearin. 1.72 Dispersion 1, or any of 1.1-1.71, wherein the dispersion does not comprise one or more of acetyl tributyl citrate, coco-caprylate/caprate, diisostearyl malate, heptyl undecylenate, isododecane, octyldodecanol, octyldodecanoyl oleate, jojoba esters, hydrogenated rapeseed oil, or synthetic wax, optionally wherein the dispersion does not comprise polyglyceryl-6 polyricinoleate or polyglyceryl-2 triisostearate;
1.73 Dispersion 1, or any of 1.1-1.72, wherein the dispersion does not comprise caprylic/caprate triglyceride and isopropyl myristate;
1.74 Dispersion 1, or any of 1.1-1.72, wherein the dispersion does not comprise coco-caprylate/caprate;
1.75 Dispersion 1, or any of 1.1-1.74, wherein the dispersion does not comprise hydrocarbons (e.g., isododecane or isohexadecane);
1.76 Dispersion 1, or any of 1.1-1.75, wherein the dispersion does not comprise diisostearyl malate and octyldodecanol;
1.77 Dispersion 1, or any of 1.1-1.76, wherein the dispersion does not comprise stearoyl glutamic acid, or isopropyl titanium triisostearate;
1.78 Dispersion 1, or any of 1.1-1.77, wherein the pigment is milled to a grind greater than or equal to a Hegman grind of 6, e.g., a Hegman grind of 6 to 8, or 6 to 7, or 7 to 8, or about 6, or about 7, or about 8; 1.79 Dispersion 1, or any of 1.1-1.78, wherein the dispersion is homogenous, e.g., the dispersion is high-sped to achieve homogenization;
1.80 Dispersion 1, or any of 1.1-1.79, wherein the dispersion is physically stable, e.g., the dispersion remains homogenous without the formation of phases, precipitates, agglomerates, lumps, or other inhomogeneities, and/or, the viscosity of the dispersion does not change significantly (e.g., less than 5%);
1.81 Dispersion 1, or any of 1.1-1.80, wherein the dispersion has a pigment wet out time of less than 250 seconds, or less than 150 seconds, or less than 100 seconds, or less than 85 seconds, or less than 60 seconds, or less than 45 seconds, or less than 35 seconds, or less than 25 seconds;
1.82 Dispersion 1, or any of 1.1-1.81, wherein the pigment is the only ingredient in the dispersion which has a color affecting the color of the dispersion (e.g., all other ingredients are colorless or do not impart any effect on the color of the dispersion);
1.83 Dispersion 1, or any of 1.1-1.82, wherein the dispersion achieves a Hegman grind of at least 4 after one hour of high-speed mixing, e.g., a Hegman grind of at least 5 or at least 6, after one hour of high-speed mixing;
1.84 Dispersion 1, or any of 1.1-1.83, wherein the dispersion is pourable (e.g., viscosity of less than 5 Pa*s) or heavy pourable (e.g., viscosity of greater than 5 and less than 20 Pa*s) after high-speed mixing and 1 to 3 passes on a roll mill, preferably pourable (e.g., preferably 0.5 to 5 Pa*s);
1.85 Dispersion, 1 or any of 1.1-1.84, wherein the dispersion has a peak hold viscosity of less than 20 Pa*s at 20/s, e.g., less than 18, or less than 15, or less than 12, or less than 10, or less than 8, or less than 6, or less than 5, or less than 4, or less than 3, or less than 2, or less than 1, or less than 0.5, Pa*S at, at 20/s;
1.86 Dispersion 1, or any of 1.1-1.85, wherein the dispersion is not a shear-thinning dispersion;
1.87 Dispersion 1, or any of 1.1-1.86, wherein the dispersion has a light skin feel;
1.88 Dispersion 1, or any of 1.1-1.87, wherein the dispersion is shelf-stable (e.g., for at least 2 weeks, or at least 3 weeks, or at least 4 weeks, or at least 6 weeks, at 40° C.), e.g., wherein the dispersion does not show signs of syneresis, precipitation, settling, or phase separation;
1.89 Dispersion 1, or any of 1.1-1.88, wherein the dispersion shows good spreadability;
1.90 Dispersion 1, or any of 1.1-1.89, wherein the dispersion is suitable for formulation into emulsion compositions.

Polyglycerols are oligomers of glycerol having the following structure:

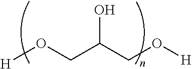

wherein n is an integer from 2-20 (e.g., 2-10 or 2-6), for example n is 2, 3, 4, 5, or 6.

Polyglycerols are an example of polyhydroxylated polyether polymers. Polyglycerols (also known as polyglycerins) are generally referred to in the form “Polyglycerol-X” wherein X is the integer n in the above formula, thus the number X refers to the total number of glycerol moieties in the molecule. For example, polyglycerol-3 is a trimer of the above formula wherein n is 3. Alternatively, they can be referred to using a numerical prefix, such as diglycerol, triglycerol, tetraglycerol etc.

A polyglycerol will have n+2 hydroxy groups, each of which can form an ester with a fatty acid. Thus, for example, polyglycerol-3, which has an n value of 3, has five hydroxy groups. Polyglycerols may be mono-esterified, typically on one of the terminal (primary aliphatic) hydroxy groups. Polyglycerols may also be diesterified, e.g., on both of the terminal (primary aliphatic hydroxy groups), or polyesterified, e.g., on the terminal hydroxy groups and one or more of the internal (secondary hydroxy groups). An ester of polyglycerol may be referred to as a polyglyceryl compound, i.e., exchanging the -ol suffix for an -yl suffix. For example, esters of polyglycerol-3 may be referred to as polyglyceryl-3 esters.

A polyesterified polyglycerol may be a mixture of esters varying in both which of the hydroxy groups is esterified and how many hydroxy groups are esterified. Polyglycerols, especially larger polyglycerols (e.g., n>2) are typically made under conditions that provide a distribution of chain lengths, such that, for example polyglycerol-3 might be a mixture of polyglycerols having an average n value of 3 (e.g., number average). Thus, while “polyglycerol-3” might have a majority of the trimer wherein n=3, there could also be small amounts (e.g., <10% by weight) of other chain lengths (such as n=5, n=4, n=2, or n=1). Likewise, esterification, especially partial esterification of a larger polyglycerol, may result in a mixture of compounds with slight variation in the ester content (e.g., polyglycerol-3 with >90% penta-esterified compound, but with small amounts of tetra-esterified, tri-esterified, or di-esterified compound).

Polyglycerols may be esterified to any fatty acid or combination of fatty acid. The fatty acids may be discretely identified, or may represent “mixed esters” which are defined by their source or components acids. Suitable fatty acids include, but are not limited to, stearic acid, isostearic acid, lauric acid, oleic acid, linoleic acid, linolenic acid, capric acid, caprylic acid, myristic acid, ricinoleic acid, behenic acid, palmitic acid, isopalmitic acid, sebacic acid, and undecylenic acid. Discrete esters are obtained by reacting a particular polyglycerol with a single particular fatty acid (or synthetic equivalent thereof) to obtain monoesters, diesters, triesters, or mixtures, etc., by controlling the molar ratio of polyglycerol to fatty acid.

Mixed esters of polyglycerols can also be formed by reacting a polyglycerol with two different fatty acids (or synthetic equivalents thereof), either together or sequentially. The molar ratio of the polyglycerol and the fatty acids can be controlled to arrive at product having particular extents of esterification to each acid, particularly if the esterifications are done sequentially. Thus, for example, polyglycerol-3 can be esterified with two molar equivalents of isostearic acid (or a synthetic equivalent thereof), to form a diisostearoyl polyglycerol-3 ester. Such a compound will primarily be esterified on the two terminal positions with the three internal hydroxy groups free. The remaining hydroxy groups can then be esterified with a second fatty acid, for example, to form a diester.

Alternatively, polyglyceryl fatty acid esters may be formed by reacting a natural mixture of fatty acids with a polyglycerol to form mixed esters whose composition may vary with the particular source of fatty acids used. For example, “olive oil polyglyceryl-3 esters” is a mixture of esters derived from olive oil (a mixture of triglycerides) and polyglycerol-3, for example, made by performing a transesterification reaction between olive oil and polyglycerol-3, and the result is therefore a mixture of polyglyceryl-3 esters having approximately the fatty acid component composition of olive oil (i.e., mostly oleic acid, linoleic acid, and palmitic acid esters). The exact fatty acid composition of the natural oil used in the reaction may vary depending on a variety of variables, such as the cultivar, growing conditions, and processing conditions, of the natural oil used.

While natural fatty acids may be most commonly employed in making polyglyceryl fatty acid esters, unnatural or semisynthetic fatty acids may also be used. One such fatty acid is dilinoleic acid, which is a synthetic dimer of the natural fatty acid linoleic acid:

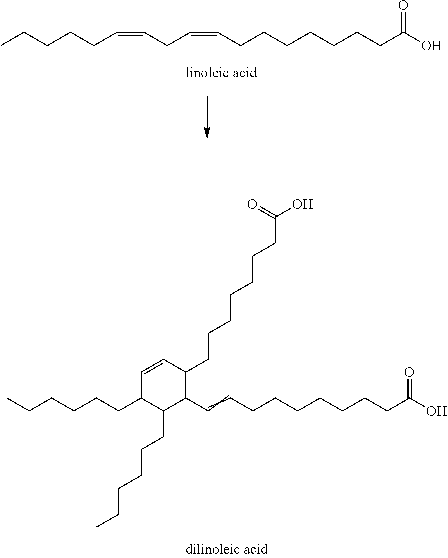 [SMILES: CCCCC/C=C\C/C=C\CCCCCCCC(=O)O ; CCCCCCC1C=CC(CCCCCCCC(=O)O)C(C=CCCCCCCCC(=O)O)C1CCCCCC]

Suitable polyglyceryl fatty acid esters are described in Fiume et al., “Safety Assessment of Polyglyceryl Fatty Acid Esters as Used in Cosmetics,” International Journal of Toxicology, 42 (Supp. 2): 5S-101S (2023). Polyglyceryl fatty acid esters are available from a variety of manufacturers, for example, diisostearoyl polyglyceryl-3 dimer dilinoleate is available as SCHERCEMOL™ PDD ester from Lubrizol, and as ISOLAN® PDI from Evonik, while triisostearoyl polyglyceryl-3 dimer dilinoleate is available as SCHERCEMOL™ PTID ester from Lubrizol.

Once the pigment is incorporated into the liquid carrier, with or without dispersants, it may then be grinded through mechanical action, for example in a media mill or three-roll mill (commonly used for color cosmetics). Grind is achieved when the pigment is the desired particle size.

Several factors are considered crucial in evaluating the performance of a pigment dispersion: wet-out time (fast wet-out being preferable), ability to undergo high-speed grinding, the amount of milling required to achieve the desired particle size and color strength; and the final viscosity of the dispersion.

Pigment wet-out is defined as the time it takes for a dry pigment to be incorporated into a liquid vehicle. A poor wet-out would display as pigment sitting on top of the liquid carrier despite high-speed mixing. In this scenario, the operator must add pigment slowly to avoid choking the mixer. The operator is required to monitor the mixer until the pigment is incorporated. For a poor wet-out, the pigment would require more time and mechanical action to mix, even before grinding can begin. An excellent wet-out would look similar to a simple dilution, where the pigment immediately mixes into the vehicle with very little time spent sitting on the surface. This would mean that the dispersion requires less pre-mixing time and operator monitoring.

Pigment color can be heavily dependent on particle size and processing. Pigment strength is typically maximized at a certain particle (agglomerate) size. Additional processing may be detrimental to the color strength, or may shift the color space (hue) with no opportunity for recovery. Because of this, it is crucial to disperse pigment in a vehicle that allows for consistent and efficient pigment grinding.

Final viscosity of a pigment dispersion is more dependent on pigment and carrier chemical properties than on dispersion quality. If a pigment is not properly dispersed, the dispersion may appear lumpy, or change viscosity over time due to pigment agglomeration or settling.

Any of the aforementioned dispersions may further comprise any one or more cosmetically acceptable ingredients. Such additional ingredients may include one or more of dispersants, surfactants, emulsifiers, emollients, humectants, preservatives, stabilizers, rheological additives, antioxidants, thickeners, and hydrocarbons. In some embodiments, the dispersions may be free of one or more of such categories of ingredients, or the dispersions may be free of all such additional ingredients.

The dispersions of the present disclosure are generally prepared by combining the polyglyceryl fatty acid ester, and any other fatty acid esters, and any other ingredients (e.g., dispersant, if applicable) using a high-speed mixer, and then the pigment is added while under agitation. The mixture is then high-speed mixed for a set amount of time, and then may be further processed through a three-roll mill until the desired grind is achieved. In some embodiments, desired grind is considered achieved once it reaches a 6 on the Hegman scale (˜25 microns), and the number of passes required to achieve that grind is recorded.

Suitable pigments according to the present disclosure include, but are not limited to, Red 4, Red 4 Lake, Red 6, Red 6 Lake, Red 7, Red 7 Lake, Red 17, Red 21, Red 21 Lake, Red 22, Red 22 Lake, Red 27, Red 27 Lake, Red 28, Red 28 Lake, Red 30, Red 30 Lake, Red 31, Red 33, Red 33 Lake, Red 34, Red 34 Lake, Red 36, Red 36 Lake, Red 40, Red 40 Lake, Blue 1, Blue 1 Lake, Blue 4, Yellow 5, Yellow 5 Lake, Yellow 6, Yellow 6 Lake, Yellow 7, Yellow 7 Lake, Yellow 8, Yellow 10, Yellow 10 Lake, Yellow 11, Green 3, Green 3 Lake, Green 5, Orange 5, Orange 5 Lake, Orange 10, Orange 10 Lake, Orange 11, Violet 2, red iron oxide, yellow iron oxide, black iron oxide, brown iron oxide, ultramarine blue, ultramarine pink, ultramarine violet, manganese violet, chromium oxide green, chromium hydroxide green, ferric ferricyanide ferric ammonium ferricyanide/iron blue, titanium dioxide, zinc oxide, bismuth oxychloride, mica, carbon black, black 2, black 3, aluminum powder, annatto, bronze powder, caramel, carmine, β-carotene, copper powder, black PN lake, triphenylmethane, erythrosine lake, Lavanya™ evelyn lake, Lavanya revolutum™, Lavanya zuni™, Lavanya decorum™ and Lavanya Belmont™, or any combination thereof.

In a second aspect, the present disclosure further provides cosmetic and personal care compositions comprising any of Dispersion 1 et seq., including, but not limited to, soaps (liquid or solid), body washes, skin and hair cleansers, skin creams and lotions (e.g., facial creams and lotions, face oils, eye cream, other anti-wrinkle products), ointments, sunscreens, moisturizers, hair shampoos and/or conditioners, deodorants, antiperspirants, other conditioning products for the hair, skin, and nails (e.g., shampoos, conditioners, hair sprays, hair styling gel, hair mousse), decorative cosmetics (e.g., nail polish, eye liner, mascara, lipstick, foundation, concealer, blush, bronzer, eye shadow, lip liner, lip balm,) and dermocosmetics. In some embodiments, the cosmetic and personal compositions have an NOI of from 0.5 to 1.0, e.g., 0.6 to 1.0, or 0.7 to 1.0, or 0.8 to 1.0, or 0.9 to 1.0, or about 1.0.

Cosmetic and personal care compositions may comprise from 0.01 to 90% by weight of the Dispersion 1 et seq., for example, 0.01 to 80%, or 0.01 to 70%, or 0.01 to 60%, or 0.01 to 50%, or 0.01 to 40%, or 0.01 to 30%, or 0.01 to 20%, or 0.01 to 10%, or 0.01 to 5%, or 0.1 to 80%, or 0.1 to 70%, or 0.1 to 60%, or 0.1 to 50%, or 0.1 to 40%, or 0.1 to 30%, or 0.1 to 20%, or 0.1 to 10%, or 0.1 to 5%, or 1 to 60%, or 1 to 50%, or 1 to 40%, or 1 to 30%, or 1 to 20%, or 1 to 10%, or 1 to 5%, or 5% to 50%, or 5% to 40%, or 5% to 30%, or 5% to 20%, or 5% to 10%, or 10 to 50%, or 10 to 40%, or 10 to 30%, or 10 to 20%, by weight of the composition.

In another embodiment of the second aspect, the present disclosure further provides industrial compositions comprising any of Dispersion 1 et seq., including, but not limited to, coatings (e.g., paints), plastics, films, composites, adhesive, caulks, sealants, and lubricants.

In another embodiment of the second aspect, the present disclosure further provides pharmaceutical compositions comprising any of Dispersion 1 et seq., including, but not limited to, sunscreens, lotions, ointments, or other medicated skin care compositions.

Finished goods manufacturers of cosmetic and personal care products seeking to include the natural content of their formulations can utilize the present invention to achieve a higher NOI for their finished products. The main limitation of NOI for cosmetic color dispersions of the present invention lies within the pigments themselves. Inorganic pigments of mineral origin, for example, iron oxides, ultramarines, mica, or titanium dioxide, generally hold an NOI of 1. Organic pigments, on the other hand, such as those subject to FDA batch certification, hold an NOI of 0. Different pigments capture different color spaces. For a formulator seeking a specific shade in their final product, inorganic pigments alone may not be satisfactory. Thus, a certain percentage of their formulation must always be allotted for ingredients with NOI equal to 0. For a cosmetic dispersion, it is most helpful if the ingredients can contribute some natural origin content, instead of entirely lending to the synthetic portion of the formula.

The present disclosure allows for the carrier vehicle to contribute a NOI of up to 1.0. Additionally, it allows for the use of untreated pigments, whether inorganic or organic in nature. Many pigment surface treatments are not naturally-derived, and thus lend themselves to the synthetic portion of a finished formula. Examples of these include silicone, alkyl silane, amino acid, amino-functional silane, ethoxylated polymers, acrylates, and metal soap surface treatments. This is especially true for pigment surface treatments used to compatibilize pigments with the silicone phase of a formulation. Untreated pigments would not contribute that surface treatment to the ingredients list, thereby preventing that additional synthetic portion and leaving more flexibility for the final formulation to be of natural origin.

Unless otherwise indicated, it is to be understood that the terminology used herein is for the purpose of describing particular embodiments only and is not intended to be limiting. In this specification and in the claims that follow, reference will be made to a number of terms, which shall be defined to have the definitions set forth below.

As used herein, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, the phrase “having the formula” or “having the structure” is not intended to be limiting and is used in the same way that the term “comprising” is commonly used.

The term “alkyl” as used herein refers to a monovalent or bivalent, branched or unbranched saturated hydrocarbon group having from 1 to 22 carbon atoms, typically although, not necessarily, containing 1 to about 12 carbon atoms, such as methyl, ethyl, n-propyl, isopropyl, n-butyl, isobutyl, t-butyl, pentyl, hexyl, heptyl, octyl, nonyl, decyl, undecyl, dodecyl, and any isomers thereof.

The term “C12-15 alkyl benzoate” is an INCI-recognized name for a mixture of esters of benzoic acid with C12-C15 alcohols. It is available from a variety of manufacturers, such as TEGOSOFT™ from Evonik, and CETIOL® from BASF.

Where a range is recited, such as 0-10 or 1-7, the range embraces all integer values within the range, as well as integer subranges. Thus, the range 0-10 includes 0-9, 0-8, 0-7, 0-6, 0-5, 0-4, 0-3, 0-2, 1-10, 1-9, 1-8, 1-7, 1-6, 1-5, 1-4, 1-3, 1-2, 1-9, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 4-4, 4-3, 5-10, 5-9, 5-8, 5-7, 5-6, 6-10, 6-9, 6-8, 6-7, 7-10, 7-9, 7-8, 8-10, 8-9, 9-10, 0, 1, 2, 3, 4, 5, 6, 7, 8, 9 and 10.

All percentages used herein, unless otherwise indicated, are by weight.

All ratios used herein, unless otherwise indicated, are by molarity.

Having been generally described herein, the following nonlimiting examples are provided to further illustrate the invention of the present disclosure.

### EXAMPLES

In a search for liquid mediums that offer these benefits of naturally-derivation, light skin feel, exceptional dispersion characteristics and color development, low viscosity, shelf stability, oil-free, good spreadability, and suitability for emulsion formulations, a wide variety of medium-to-low polarity liquids suitable for use in color cosmetics were examined for dispersions having the desired dispersion properties. These materials included esters, liquid polymers, naturally-derived oils, ethers, and combinations thereof. These liquid carrier vehicles were examined with and without the presence of naturally-derived wetting and dispersing additives for their ability to wet and disperse pigment under high shear conditions to a Hegman grind of 6 or more, using a high-speed overhead mixer with high-shear blade and a three-roll mill.

### Example 1

In this example, several carriers commonly utilized in currently available pigment dispersions on the cosmetic market are included in the testing, such as isononyl isononanoate, C12-C15 alkyl benzoate, caprylic/capric triglyceride, polyglyceryl-6 polyricinoleate, jojoba oil, and sunflower oil. The following dispersion compositions are prepared and evaluated:

TABLE 1
(Preparation) (values are in weight %)

| Dispersion: | 1 | 2 | 3 | 4 | 5 | 6 | 7 | 8 | 9 | 10 | 11 | 12 | 13 |
|---|---|---|---|---|---|---|---|---|---|---|---|---|---|
| Isononyl | 40 | 38.5 | | | | | 20 | | | | | | |
| Isononanoate | | | | | | | | | | | | | |
| C12-15 Alkyl | | | 40 | 38.5 | | | | | | | | | |
| Benzoate | | | | | | | | | | | | | |
| Caprylic/Capric | | | | | 40 | 38.5 | | | | | | | |
| Triglyceride | | | | | | | | | | | | | |
| Polyglyceryl-6 | | | | | | | 20 | 40 | 38.5 | | | | |
| Polyricinoleate | | | | | | | | | | | | | |
| Jojoba Oil | | | | | | | | | | 40 | 38.5 | | |
| Sunflower Oil | | | | | | | | | | | | 40 | 38.5 |
| Polyhydroxystearic | | 1.5 | | 1.5 | | 1.5 | | | 1.5 | | 1.5 | | 1.5 |
| Acid | | | | | | | | | | | | | |
| Titanium Dioxide | 60 | 60 | 60 | 60 | 60 | 60 | 60 | 60 | 60 | 60 | 60 | 60 | 60 |

Each composition is prepared by mixing the major liquid components (fatty acid esters or other carrier vehicle), followed by adding any dispersants (e.g., polyhydroxystearic acid) and the pigment (titanium dioxide) to form a premix dispersion. Observations are made of the premix dispersion to evaluate how well the vehicle wets and disperses the pigment particles with low-shear overhead mixing. The premix dispersion is then submitted to high-shear overhead mixing for thirty minutes, or until the material is no longer fluid enough to mix, resulting in the high-shear mixed dispersion, in order to evaluate the high-speed grind. Observations are made of the high-shear mixed dispersion to evaluate how well the vehicle has wet and dispersed the pigment particles. The high-shear mixed dispersion is then passed through a three-roll mill to form the final dispersion. The number of passes needed through the mill to achieve the desired final grind is determined, and the final grind (Hegman scale) is determined. Premix compositions which achieve insufficient initial wetting are discarded without proceeding to grinding and milling. The final dispersion is then evaluated under accelerated aging conditions of 40° C. for 2 weeks, 4 weeks, and 6 weeks, for signs of syneresis and separation. The following scale is used for evaluating stability on two phenomena, syneresis and settling. In the evaluation Tables below, the score is shown as the Syneresis Score/Settle Score (e.g., a 4/3 score means some syneresis with soft settle):

| Syneresis Score | | Settling Score | |
|---|---|---|---|
| Score | Description | Score | Description |
| 1 | No syneresis | 1 | No settling |
| 2 | Very slight syneresis | 2 | Slight soft settle |
| 3 | Slight syneresis | 3 | Soft settle |
| 4 | Some syneresis | 4 | Hard packing (hard settle) |
| 5 | Significant syneresis | | |
| 6 | Major syneresis | | |
| 7 | Complete separation | | |
| | (complete syneresis) | | |

It is understood that a dispersion may undergo either syneresis, settling, or both, and any combination of these events is indicative of decreasing stability of the dispersion.

The results for the dispersions of Table 1 are shown in Table 2 below (I=insufficient; S=sufficient; E=exceptional):

TABLE 2
(Evaluation)

| Dispersion: | 1 | 2 | 3 | 4 | 5 | 6 | 7 | 8 | 9 | 10 | 11 | 12 | 13 |
|---|---|---|---|---|---|---|---|---|---|---|---|---|---|
| Premix | I | I | I | I | I | I | S | S | S | I | I | I | I |
| Dispersion | | | | | | | | | | | | | |
| Observations | | | | | | | | | | | | | |
| High-Shear | N/A | N/A | N/A | N/A | N/A | N/A | 2 | 5 | 5.5 | N/A | N/A | N/A | N/A |
| Mixed | | | | | | | | | | | | | |
| Dispersion | | | | | | | | | | | | | |
| Grind | | | | | | | | | | | | | |
| Roll Mill | N/A | N/A | N/A | N/A | N/A | N/A | 1 | 1 | 1 | N/A | N/A | N/A | N/A |
| Passes | | | | | | | | | | | | | |
| Final | N/A | N/A | N/A | N/A | N/A | N/A | 7 | 6 | 6.5 | N/A | N/A | N/A | N/A |
| Dispersion | | | | | | | | | | | | | |
| Grind | | | | | | | | | | | | | |
| 40° C., 2 weeks | | | | | | | 4/1 | 4/1 | 4/1 | | | | |
| 40° C., 4 weeks | | | | | | | 4/1 | 3/1 | 3/1 | | | | |
| 40° C., 6 weeks | | | | | | | 7/1 | 3/1 | 3/1 | | | | |

The results show that dispersions comprising simple fatty acid mono esters (isononyl isononanoate or C12-C15 alkyl benzoate) or triglycerides (caprylic/capric) provide insufficient wetting at the premix stage (for 60% pigment), even with PHSA dispersant, and are unsuitable for further evaluation. Dispersions 7, 8, and 9, having the polyglyceryl fatty acid ester (with or without PHSA dispersant), are suitable for grinding and milling, but result in dispersions having inadequate stability, exhibiting significant separation.

### Example 2

The procedure of Example 1 is followed to prepare the following additional dispersions, each also utilizing common cosmetic ingredients:

TABLE 3
(Preparation) (values are in weight %)

| Dispersion: | 14 | 15 | 16 | 17 | 18 | 19 | 20 | 21 |
|---|---|---|---|---|---|---|---|---|
| Olive Oil PEG-7 Esters | 40 | 38.5 | | | | | | |
| Decyl Oleate | | | 40 | 38.5 | | | | |
| Polyglyceryl-3 Laurate | | | | | 40 | 38.5 | | |
| Isostearyl Isostearate | | | | | | | 40 | 38.5 |
| Polyhydroxystearic Acid | | 1.5 | | 1.5 | | 1.5 | | 1.5 |
| Titanium Dioxide | 60 | 60 | 60 | 60 | 60 | 60 | 60 | 60 |

The results for the dispersions of Table 3 are shown in Table 4 below (J=insufficient; S=sufficient; E=exceptional):

TABLE 4
(Evaluation)

| Dispersion: | 14 | 15 | 16 | 17 | 18 | 19 | 20 | 21 |
|---|---|---|---|---|---|---|---|---|
| Premix | S | E | I | S | I | S | I | S |
| Dispersion | | | | | | | | |
| Observations | | | | | | | | |
| High-Shear | 4 | 5.5 | N/A | 2.5 | N/A | 5 | N/A | 2 |
| Mixed | | | | | | | | |
| Dispersion Grind | | | | | | | | |
| Roll Mill Passes | 1 | 1 | N/A | 1 | N/A | 1 | N/A | 1 |
| Final Dispersion | 6 | 6 | | 7 | | 6 | | 7 |
| Grind | | | | | | | | |
| 40° C., 2 weeks | 1/1 | 1/1 | | 6/3 | | 1/1 | | 5/1 |
| 40° C., 4 weeks | 1/1 | 2/1 | | 5/3 | | 1/1 | | 5/3 |
| 40° C., 6 weeks | 2/1 | 2/1 | | 7/3 | | 4/1 | | 5/3 |
| 25° C., 3 months | 1/1* | 1/1 | | 7/4 | | 1/1 | | 6/3 |

*Cracking/leaching observed

The results show that the dispersion compositions 14, 15, 17, and 21 provide good initial wetting and acceptable final grinds of Hegman 6 and 7, but the compositions are mostly unstable on storage at either 25° C. or 40′C. Dispersion composition 19 was very slow to wet out pigment and experienced excess heat on the overhead mixer, resulting in a mostly stable dispersion that was much higher in viscosity than other trials. Overall, the dispersion with olive oil PEG-7 esters (15) has good properties, but this agent has a low natural origin index and was found to cause emulsion stability issues in end-applications due to its water-solubility. The dispersions with decyl oleate (17) or isostearyl isostearate (21) achieved grind quickly on the roll mill, but on their own were not sufficient to keep pigment suspended and remain homogeneous dispersions. The dispersion with polyglyceryl-3 laurate (19) did not wet pigment efficiently but dispersed well and remained stable.

### Example 3

The procedure of Example 1 is followed to prepare the following additional dispersions, each also utilizing common cosmetic ingredients:

TABLE 5
(Preparation) (values are in weight %)

| Dispersion: | 22 | 23 | 24 | 25 | 26 | 27 |
|---|---|---|---|---|---|---|
| Diisostearoyl Dimer Dilinoleate | 40 | 38.5 | | | | |
| Diisostearoyl Polyglyceryl-3 | | | 40 | 38.5 | | |
| Dimer Dilinoleate | | | | | | |
| Triisostearoyl Polyglyceryl-3 | | | | | 40 | 38.5 |
| Dimer Dilinoleate | | | | | | |
| Polyhydroxystearic Acid | | 1.5 | | 1.5 | | 1.5 |
| Titanium Dioxide | 60 | 60 | 60 | 60 | 60 | 60 |

The results for the dispersions of Table 5 are shown in Table 6 below (J=insufficient; S=sufficient; E=exceptional):

TABLE 6
(Evaluation)

| Dispersion: | 22 | 23 | 24 | 25 | 26 | 27 |
|---|---|---|---|---|---|---|
| Premix | I | S | I | S | I | E |
| Dispersion | | | | | | |
| Observations | | | | | | |
| High-Shear | N/A | 3 | N/A | 2 | N/A | dirty 6 |
| Mixed | | | | | | |
| Dispersion Grind | | | | | | |
| Roll Mill Passes | N/A | 1 | N/A | 1 | N/A | 1 |
| Final Dispersion | N/A | 6.5 | N/A | 7 | N/A | 7 |
| Grind | | | | | | |
| 40° C., 2 weeks | | 1/1 | | 1/1 | | 1/1 |
| 40° C., 4 weeks | | 2/1 | | 1/1 | | 1/1 |
| 40° C., 6 weeks | | 4/3 | | 1/1 | | 1/1 |

The results show that the dispersion compositions 23, 25, and 27, provide good initial wetting and acceptable final grinds of Hegman 6.5 and 7, while the dispersion compositions 25 and 27 also show excellent stability. However, composition 23 exhibited instability at 40° C. over 6 weeks, and compositions 25 and 27 were expensive compositions with very high viscosity, and high tack.

### Example 4

The results from Examples 1-3, however, suggested the possibility that by combining a monoester (such as decyl oleate, isostearyl isostearate, or polyglyceryl-3 laurate), with a diester (such as diisostearoyl dimer dilinoleate or diisostearoyl polyglyceryl-3 dimer dilinoleate) or with a triester (such as triisostearoyl polyglyceryl-3 dimer dilinoleate or caprylic/capric triglyceride) some very good properties might be obtained.

The procedure of Example 1 is followed to prepare the following additional dispersions, each also utilizing common cosmetic ingredients:

TABLE 7
(Preparation) (values are in weight %)

| Dispersion: | 28 | 29 | 30 | 31 | 32 | 33 | 34 |
|---|---|---|---|---|---|---|---|
| Isononyl Isononoate | 20 | | | | | | |
| Caprylic/Capric | | 20 | 19.25 | 19.25 | | | |
| Triglyceride | | | | | | | |
| Isostearyl Isostearate | | | | | 19.25 | | |
| Decyl Oleate | | | | | | 20 | 25 |
| Olive Oil Polyglyceryl-3 | | | | 19.25 | | | |
| Esters | | | | | | | |
| Polyglyceryl-3 Laurate | 20 | | 19.25 | | | | |
| Triisostearoyl Polyglyceryl- | | 20 | | | 19.25 | 18.5 | 12.9 |
| 3 Dimer Dilinoleate | | | | | | | |
| Polyhydroxystearic Acid | | | 1.5 | 1.5 | 1.5 | 1.5 | 1.5 |
| Trihydroxystearin | | | | | | | 0.6 |
| Titanium Dioxide | 60 | 60 | 60 | 60 | 60 | 60 | 60 |

The results for the dispersions of Table 3 are shown in Table 4 below (J=insufficient; S=sufficient; E=exceptional):

TABLE 8
(Evaluation)

| Dispersion: | 28 | 29 | 30 | 31 | 32 | 33 | 34 |
|---|---|---|---|---|---|---|---|
| Premix | S | S | S | S | E | S | E |
| Dispersion | | | | | | | |
| Observations | | | | | | | |
| High-Shear | 0 | 4 | 4 | 3 | 7 | 2 | 7 |
| Mixed | | | | | | | |
| Dispersion Grind | | | | | | | |
| Roll Mill Passes | 1 | 2 | 1 | 2 | 0 | 1 | 0 |
| Final Dispersion | 7 | 6.5 | 6.5 | 7 | 7 | 6 | 7 |
| Grind | | | | | | | |
| 40° C., 2 weeks | 4/1 | 1/3 | 1/1 | 2/1 | 1/3 | 1/1 | 1/1 |
| 40° C., 4 weeks | 3/1 | 1/10 | 2/1 | 3/1 | 1/3 | 1/1 | 1/1 |
| 40° C., 6 weeks | 4/1 | 1/10 | 2/1 | 1/1 | 1/2 | 1/3 | 1/1 |

The results show that dispersions 30, 31 and 34 provide the best results. They each provide good initial wetting and acceptable final grinds of Hegman 6-7, and excellent stability.

Overall, the data demonstrates that the best dispersion properties of each class of monoesters, diester, or triesters, could be maintained without performance significantly suffering from the downfalls of each individual carrier vehicle.

### Example 5

Color strength is evaluated between two compositions according to the present disclosure, versus two commercial pigment dispersion formulations. Dispersion 32 and Dispersion 33 from Example 4 are compared to commercial dispersion formulations which do not have polyglyceryl fatty acid ester vehicles. Commercial Dispersion A has 70% titanium dioxide dispersed in isononyl isononanoate and isopropyl myristate, aided and stabilized with stearalkonium hectorite, isopropyl titanium triisostearate, propylene carbonate and polyhydroxystearic acid. Commercial Dispersion B has 60% titanium dioxide dispersed in caprylic/capric triglyceride and isopropyl myristate, aided and stabilized with stearoyl glutamic acid, stearalkonium hectorite, trihydroxystearin, and propylene carbonate. For the purpose of color development analysis, color strength is evaluated on a 1:1 dry pigment basis. The results are shown in the following table:

TABLE 9

| Dispersion | Color strength |
|---|---|
| Commercial Dispersion A | 78% |
| Commercial Dispersion B | 81% |
| Dispersion 32 | 100% |
| Dispersion 33 | 100% |

Each dispersion was evaluated at 8% in a green tint base and compared to each other. One formula was chosen to be “standard” and all other strength numbers are measured relative to it. The batch with color strength 100% is “standard”.

The results show that the best color development is obtained using the compositions according to the present disclosure.

It is particularly noteworthy that these favorable results were achieved without the addition of natural oils, petroleum-based materials, silicone or silicone-derivatives, or alcohols.

The dispersions according to the present disclosure thus achieve the many benefits described herein: light skin feel (emolliency), exceptional pigment particle (agglomerate) size reduction with reasonable processing times, exceptional color development, low viscosity, shelf stability, “oil-free” formulations, good spreadability, suitability for emulsion formulations, and up to 100% naturally-derived formulations.

While these examples, and the discoveries described herein, are presented using cosmetic and personal care applications, the unexpected findings are applicable to pigment dispersions and dispersions generally for use in industrial markets, such as coatings, plastics, films, composites, adhesives, caulks, sealants, and lubricants. An example of a coatings application would be pigment dispersions formulated to increase sustainability while delivering low viscosity, stable pigment dispersions that are volatile-free and broadly compatible with various film-formers. Its findings are also applicable to pigment dispersions and dispersions generally for use in the pharmaceutical market. An example of a pharmaceutical application would be mineral dispersions for use as UV filters in a sunscreen.

I/We claim:

1. A pigment dispersion a pigment dispersion comprising at least one polyglyceryl fatty acid ester.

2. The dispersion of claim 1, wherein the polyglyceryl fatty acid ester is an ester of a polyglycerol of the following formula, wherein n is an integer from 2 to 20;

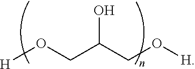

3. The dispersion of claim 2, wherein n is an integer selected from 2, 3, 4, 5, and 6.

4. The dispersion of claim 2 or 3, wherein the polyglyceryl fatty acid ester is a polyglyceryl-3 fatty acid ester.

5. The dispersion of any one of claims 1-4, wherein the polyglyceryl fatty acid ester is a stearate, isostearate, laurate, oleate, linoleate, linolenate, caprate, caprylate, myristate, ricinoleate, behenate, palmitate, isopalmitate, sebacate, undecylenate, or dilinoleate ester.

6. The dispersion of any one of claims 1-5, wherein the polyglyceryl fatty acid ester is selected from any of the following or a combination thereof: Diisostearoyl Polyglyceryl-3 Dimer Dilinoleate, Polyglyceryl-3 Behenate, Polyglyceryl-3 Caprate, Polyglyceryl-3 Caprylate, Polyglyceryl-3 Dicaprate, Polyglyceryl-3 Dicitrate/Stearate, Polyglyceryl-3 Di-Hydroxystearate, Polyglyceryl-3 Diisostearate, Polyglyceryl-3 Dioleate, Polyglyceryl-3 Distearate, Polyglyceryl-3 Isostearate, Polyglyceryl-3 Laurate, Polyglyceryl-3 Myristate, Polyglyceryl-3 Oleate, Polyglyceryl-3 Palmitate, Polyglyceryl-3 Pentacaprylate/Caprate, Polyglyceryl-3 Pentaricinoleate, Polyglyceryl-3 Ricinoleate, Polyglyceryl-3 Stearate, Polyglyceryl-3 Stearate SE, Polyglyceryl-3 Triisostearate, and Triisostearoyl Polyglyceryl-3 Dimer Dilinoleate.

7. The dispersion of claim 1, wherein the polyglyceryl fatty acid ester is selected from Diisostearoyl Polyglyceryl-3 Dimer Dilinoleate, and Triisostearoyl Polyglyceryl-3 Dimer Dilinoleate.

8. The dispersion of any one of claims 1-7, wherein the dispersion further comprises one or more other fatty acid esters.

9. The dispersion of claim 8, wherein the one or more other fatty acid esters comprise one or more of isononyl isononanoate, decyl oleate, isostearyl laurate, caprylic/capric triglyceride, diisostearoyl dimer dilinoleate, or a C12-C15 fatty acid benzoate.

10. The dispersion of claim 8, wherein the one or more other fatty acid esters comprise one or more of isononyl isononanoate and decyl oleate.

11. The dispersion of claim 1, wherein the dispersion comprises a combination of Diisostearoyl Polyglyceryl-3 Dimer Dilinoleate and decyl oleate.

12. The dispersion of claim 1, wherein the dispersion comprises a combination of Triisostearoyl Polyglyceryl-3 Dimer Dilinoleate and decyl oleate.

13. The dispersion of claim 1, wherein the dispersion comprises a combination of Diisostearoyl Polyglyceryl-3 Dimer Dilinoleate and isostearyl isostearate.

14. The dispersion of claim 1, wherein the dispersion comprises a combination of Triisostearoyl Polyglyceryl-3 Dimer Dilinoleate and isostearyl isostearate.

15. The dispersion of claim 1, wherein the dispersion comprises a combination of Polyglyceryl-3 Laurate, and caprylic/caprate triglyceride.

16. The dispersion of any one of claims 1-15, wherein the dispersion does not comprise one or more wetting, dispersing, and/or anti-settling aids.

17. The dispersion of any one of claims 1-15, wherein the dispersion further comprises polyhydroxystearic acid or trihydroxystearin.

18. A cosmetic or personal care compositions comprising a dispersion according to any one of claims 1-17.

19. The cosmetic or personal care composition according to claim 18, wherein the composition has a natural origin index (NOI) of from 0.5 to 1.0, e.g., 0.6 to 1.0, or 0.7 to 1.0, or 0.8 to 1.0, or 0.9 to 1.0.

20. A pharmaceutical composition comprising a dispersion according to any one of claims 1-17.
